(19) United States
(12) Patent Application Publication
(10) Pub. No.: US 2023/0405113 A1
(43) Pub. Date: Dec. 21, 2023

(54) SYSTEMS FOR BUCCAL RELEASE OF PROTEINS

(71) Applicants: Centre National de la Recherche Scientifique, Paris (FR); Universite Claude Bernard Lyon 1, Villeurbanne (FR)

(72) Inventors: Claire Monge, Nivolas-Vermelle (FR); Bernard Verrier, Mornant (FR); Anne-Lise Paris, Lyon (FR); Sofia Caridade, Lyon (FR)

(21) Appl. No.: 18/035,358

(22) PCT Filed: Nov. 12, 2021
(86) PCT No.: PCT/EP2021/081473

§ 371 Date: May 4, 2023

(30) Foreign Application Priority Data
Nov. 12, 2020 (FR) 2011592

(51) Int. Cl. A61K 39/35; A61K 9/00; A61K 47/36; A61P 37/08

(52) U.S. Cl. CPC A61K 39/35; A61K 9/006; A61K 47/36; A61P 37/08; A61K 2039/542

(57) ABSTRACT

The present invention relates to a mucoadhesive patch for buccal or sublingual release of a protein or polypeptide, said patch comprising at least 100 bilayers each composed of a layer of chitosan (CHI) and a layer of an anionic polysaccharide. The invention also relates to a method for producing such a patch.

The invention relates to a system, in the form of a mucoadhesive patch, for buccal release of a protein.

### TECHNOLOGICAL BACKGROUND

Oral administration of medicinal products is the most widely used route of administration. However, the release of proteins of therapeutic interest by this route is difficult as proteins are highly sensitive to the action of the enzymes of the gastrointestinal tract. The buccal mucosa represents an appealing alternative route of administration, as it makes it possible to avoid the first-pass effect, has low enzymatic activity and a physiological pH range, and is anatomically accessible and well vascularized. A system for buccal release could thus supply either local (mucosal) release or systemic (transmucosal) release of the protein.

There is still an unmet need for a mucoadhesive system having optimized permeation capacity, which would contribute to a significant reduction in the therapeutic dose administered as well as better control of absorption.

### SUMMARY OF THE INVENTION

To address this need, the inventors have developed a mucoadhesive patch consisting of a combination of two polysaccharides: a chitosan (CHI), and an anionic polysaccharide, such as hyaluronic acid (HyA).

The patch of the invention, which adheres to the buccal or sublingual mucosa, is called a “buccal” or “sublingual” patch.

The invention thus supplies a mucoadhesive patch intended for buccal or sublingual release of a protein or of a polypeptide, said patch comprising at least 100 bilayers each made up of a layer of chitosan (CHI) and a layer of an anionic polysaccharide having a molecular weight between 500 and 1000 kDa or a salt thereof, the layer in contact with the mucosa and the layer in contact with the buccal or sublingual environment of the patch consisting of chitosan (CHI), said protein or said polypeptide being incorporated in said patch and/or adsorbed on the surface of said patch.

In a preferred embodiment, the invention supplies a buccal or sublingual mucoadhesive patch, comprising a protein or a polypeptide, said patch comprising between 100 and 200 bilayers each made up of a layer of chitosan (CHI) and a layer of an anionic polysaccharide having a molecular weight between 500 and 1000 kDa or a salt thereof, said anionic polysaccharide being hyaluronic acid,
the layer in contact with the mucosa and the layer in contact with the buccal or sublingual environment of the patch consisting of chitosan (CHI), said protein or said polypeptide being incorporated in said patch and/or adsorbed on the surface of said patch, and intended for buccal or sublingual release.

The patch is self-supporting and dissolves in the saliva owing to the presence of enzymes capable of degrading the two polysaccharides.

The invention further relates to a method for producing said patch, comprising the steps consisting of:
i) forming a CHI/anionic polysaccharide multilayer membrane by a method of layer by layer deposition on a substrate;
ii) detaching the multilayer membrane from the substrate;
iii) bringing the multilayer membrane into contact with a protein, whereby the multilayer membrane is loaded with the protein.

The invention further relates to a patch according to the invention obtainable by a method as described here.

### LEGEND OF THE FIGURES

FIG. 1: Thickness and degradation profiles of the (CHI/HyA)₁₀₀ self-supporting membranes. (A) Membrane thickness as a function of variations in the molecular weight of HyA (660 or 1020 kDa, corresponding to HyA.LW and HyA.HW respectively) or deposition time: Short Cycles or Long Cycles. The statistical significance of the differences between two groups was determined using an ANOVA test with one controlled factor: ns, not significant; ****p<0.0001. (B) Degradation profiles of the membranes immersed in artificial saliva, expressed as percentage weight loss as a function of different production parameters of the membranes. (C) Scanning electron microscopy (SEM) images of short cycle (CHI/HyA₆₆₀)-CHI membranes, immersed in the artificial saliva for 30 min, 1 h or 3 h. Scale bar corresponding to 44 μm. (D) Images of short cycle (CHI^{FITC}/HyA₆₆₀)-CHI^{FITC} membranes obtained by confocal microscopy after 30 min, 3 h, 6 h or 24 h in the artificial saliva. The control condition corresponds to immersion of the membrane in acetate buffer for 24 h. a, b, c, d, e: surface of the membrane; lower sections: images of depthwise section (z-section) of the same membranes. Horizontal scale bar corresponds to 50 μm.

FIG. 2: The degradation products of the membranes do not display toxicity for human epithelial cells. Cellular viability after incubation of (A) HeLa cells and (B) Ho-1u-1 cells with degradation products for 24 h. Membrane: (CHI/HyA)₁₀₀ membranes; Membrane.HCl: (CHI/HyA)₁₀₀ membranes treated with hydrochloric acid (HCl). The statistical significance of the differences between two groups was determined using an ANOVA test with a single controlled factor: ns, not significant.

FIG. 3: Controlled inflammation of the sublingual mucosa by treating the membranes with HCl. The thickness of the mucosa (A) and the recruitment of the MHCII positive (MHCII+) cells (B) were evaluated in the whole mucosa (epithelium and lamina propria) after application of a membrane or of 1-fluoro-2,4-dinitrobenzene (DNFB). The native membranes (Patch) were administered for 30 min or 60 min and the membranes treated with HCl (Patch HCl) or NaCl (Patch NaCl) were applied for 30 min. Each value supplied corresponds to the mean of at least 3 measurements from 3 independent individuals (N=3). The statistical significance of the differences between two groups was determined using an ANOVA test with a single controlled factor: ns, not significant; *, p<0.05. (C) Images representing the recruitment of MHCII+ cells at the level of the mucosa, obtained by histologic labeling. Scale bar: 170 μm. (D/E/F) Levels of inflammatory cytokines in the tongue and the sublingual tissues measured 6 h after application of membranes in mice: IL-10 (D), IL-6 (E) and TNF-α (F). The statistical significance of the differences between two groups was determined using an ANOVA test with a single controlled factor: ns, not significant; *, p<0.05, ***p<0.001, ****, p<0.0001.

FIG. 4: Schematic representation of the incorporation of proteins by variation of pH in layer by layer (LbL) assembly. After functionalization by protein trapping, the LbL membrane becomes a bioactive patch, ready to be applied. CHI: chitosan, HyA: hyaluronic acid.

FIG. 5: Incorporation, and profiles of release from the membrane, of ovalbumin labeled with Alexa Fluor 647 (OVA^{Alexa F.647}). (A) Levels of incorporation of OVA^{Alexa F.647} in the (CHI/HyA)₁₀₀-CHI patch after incubation with 0.5 μg of proteins in HCl and rinsing with various buffers. (B) Profiles of release of OVA^{Alexa F.647} from the patch over a rinsing time of 2 h with various solutions. The data correspond to the mean value with standard error (±SEM) of triplicates from three independent experiments (N=3). (C, upper part) Confocal microscopy image of the surface of the patch containing OVA^{Alexa F.647} scale bar corresponding to 50 μm. (C, lower part) Depthwise optical section of the patch, along the (z) axis. The dotted lines represent the limits of the patch. (D) Fluorescence intensity of OVA^{Alexa F.647} and of the patch (CHI._{MW}^{FITC}) along the arrow shown in C (lower part).

FIG. 6: Retention time of the patch on the sublingual mucosa. (A) Staining of the (CHI/HyA)₁₀₀ patch with hematoxylin brought into contact with the sublingual mucosa, 20 minutes after administration of the patch. Scale bar corresponding to 100 μm. M=mucosa, P=Patch (B) Molecular fluorescence tomography of OVA^{AF647} in solution or incorporated in the (CHI/HyA)₁₀₀ patch. The fluorescence signal was detected 2, 10, or 30 min after administration.

FIG. 7: Tissue penetration of OVA^{AF647} after administration of the patch. Confocal microscopy of sections of mouse tongues with nuclear labeling of the cells with DAPI, 2 min after administration of OVA^{AF647} in solution or 2 min, 10 min, 30 min and 60 min after administration of OVA^{AF647} incorporated in the (CHI/HyA)₁₀₀ patch. OVA^{AF647} was observed in the keratinized layers 10 min after administration (white arrows) and in the submucosa 30 min after administration (dotted arrow). Scale bar: 20 μm. Dotted lines correspond to the limit of the mucosa.

FIG. 8: The chemo-attraction capacities of cytokine CCL20 are preserved in the patch. Chemotaxis in vitro of CCL20 released from the patch compared to CCL20 in solution. A concentration of 25 ng·mL⁻¹ was used for evaluating the migration of the DC 2.4 cells. Growth medium, salivary enzymes alone and (CHI/HyA)₁₀₀ membranes were used for the negative controls. The statistical significance of the differences between two groups was determined using an ANOVA test with one controlled factor: ns, not significant; *, p<0.05, **p<0.01.

FIG. 9: Thickness and degradation profiles of the (CHI/HyA)₁₀₀-CHI or (VIS/HyA)₁₀₀-VIS self-supporting membranes. (A) Thickness of the membranes according to the number of bilayers: 50, 100, or 200 bilayers with a final layer of HyA. Thickness of the membranes made from 100 bilayers with a final layer produced with CHI obtained from Sigma or with Viscosan® (VIS) from Flexichem. Each type of membrane was measured at least 20 times. The statistical significance between two groups was determined using an ANOVA test with a single controlled factor: ns, not significant, ****, p<0.0001. (B) Degradation profile of the membranes immersed in artificial saliva expressed as percentage weight loss as a function of different types of membranes made of CHI or of VIS. Each value given corresponds to the mean value of triplicates from three independent experiments (N=3).

FIG. 10: Incorporation and profiles of release of OVA⁶⁴⁷ from the membrane. (A) Levels of incorporation of OVA^{AF647} in the (CHI/HyA)₁₀₀-CHI patch after incubation with 0.5 μg of proteins in NaCl and rinsing with various buffers. (B) Release profiles of OVA^{AF647} from the patch over a rinsing time of 2 h with various solutions. The data correspond to the mean value of triplicates from three independent experiments. (C, upper part) Confocal microscopy image of the surface of the patch containing OVA^{AF647}, scale bar corresponding to (C, lower part) Depthwise optical section of the patch, along the (z) axis. The dotted lines represent the limits of the patch. (D) Fluorescence intensity of OVA^{AF647} and of the (CHI^{FITC}) patch along the arrow shown in Figure C (lower part).

FIG. 11: Scheme illustrating trapping of the protein or polypeptide by swelling or tightening of the multilayer membrane.

### DETAILED DESCRIPTION OF THE INVENTION

As illustrated in the examples given hereunder, the inventors have developed a mucoadhesive patch for buccal or sublingual release of a protein ensuring better control of the dose of protein administered and of its absorption by the mucosae. The inventors have also demonstrated the nontoxic character of a patch of this kind and the absence of inflammation of the mucosae after application of a patch of this kind.

The present invention therefore relates to a mucoadhesive patch intended for buccal or sublingual release of a protein or of a polypeptide, said patch comprising at least 100 bilayers each made up of a layer of chitosan (CHI) and a layer of an anionic polysaccharide having a molecular weight between 500 and 1000 kDa or a salt thereof, the layer in contact with the mucosa and the layer in contact with the buccal or sublingual environment of the patch consisting of chitosan (CHI), said protein or said polypeptide being incorporated in said patch and/or adsorbed on the surface of said patch.

### Definitions

“Patch” means a multilayer adhesive system or a multilayer adhesive membrane comprising a biologically active compound, such as a protein or a polypeptide.

“Mucoadhesive” means a patch as defined in the present application that can adhere to a mucosa, preferably a buccal or sublingual mucosa.

“Self-supporting” means a structure devoid of support, whose rigidity in itself provides its stability.

The term “subject” signifies all human persons or animals, preferably mammals, such as equines, Ovidae, bovines, dogs, cats etc.

Proteins and Polypeptides

The patch according to the invention allows release of any protein or polypeptide of interest. “Protein” means typically a polypeptide of at least 100 amino acids, or polypeptides combined together. The proteins may preferably have a molecular weight between 20 and 200 kDa. More specifically, the proteins may have a molecular weight below 70 kDa, or a higher molecular weight, for example from 100 to 200 kDa.

Among the proteins of interest, we may mention for example antibodies (such as immunoglobulins G).

The polypeptides, which may be fragments of proteins, typically have from 10 to 100 amino acids, more preferably from 20 to 80 or 20 to 60 amino acids.

In a preferred embodiment, the protein or the polypeptide is an allergen.

The allergens as defined in the present invention comprise antigens capable of stimulating an allergic reaction in a subject. The allergens may be contained in or be derived from foodstuffs such as milk, eggs, sesame, wheat, soybean, fish, seafood, peanuts, nuts. The allergens may also be contained in or be derived from nonfood products such as mites, pollen, insect bites, animal fur, wool, medicinal products, etc. The allergens are preferably polypeptides or proteins forming the whole or part of an antigen recognizable by a cell of the immune system, and with respect to which we aim to induce tolerance to the allergen.

In a preferred embodiment example, the protein to be delivered is ovalbumin. We may also mention, among the preferred food allergens, beta-lactoglobulin, alpha-lactalbumin, the caseins.

The invention thus relates to an allergen for use in the desensitization of a subject allergic to said allergen, said allergen being administered in the form of a patch as described here. Desensitization of the subject, also called allergy immunotherapy, allows the subject to become tolerant to a particular allergen in the long term.

Multilayer Patch

The patch according to the invention comprises at least 100 bilayers each made up of a layer of chitosan (CHI) and a layer of an anionic polysaccharide having a molecular weight between 500 and 1000 kDa or a salt thereof, where the layer in contact with the mucosa and the layer in contact with the buccal or sublingual environment consist of chitosan (CHI).

The patch according to the invention is formed from a multilayer adhesive membrane comprising at least 100 bilayers each made up of said chitosan and of said anionic polysaccharide where the two layers located at the two ends or at the periphery of this membrane consist of chitosan (CHI).

According to a particular embodiment of the invention, the patch comprises 100 to 200 CHI/anionic polysaccharide bilayers. According to a preferred embodiment, the patch comprises 100 or 200 CHI/anionic polysaccharide bilayers, and even more preferably 100 CHI/anionic polysaccharide bilayers.

According to another particular embodiment of the invention, the patch has a thickness from 10 to 20 μm, preferably from 11 to 19 μm, 12 to 18 μm, 13 to 17 μm, 14 to 16 μm, and even more preferably of about 15 μm.

Chitosan is a polysaccharide consisting of the random distribution of D-glucosamine bound at β-(1-4) (deacetylated unit) and N-acetyl-D-glucosamine (acetylated unit). It is produced by chemical deacetylation (in an alkaline medium) or enzymatic deacetylation of chitin, which is the component of the exoskeleton of arthropods (crustaceans) or of the endoskeleton of cephalopods (squid etc.) or of the wall of fungi. This raw material is typically demineralized by treatment with hydrochloric acid, then deproteinized in the presence of sodium hydroxide or potassium hydroxide and finally decolorized with an oxidizing agent. The degree of acetylation (DA) is the percentage of acetylated units relative to the total number of units; it can be determined by Fourier transform infrared spectroscopy (FTIR) or by titration with a strong base.

Preferably, the chitosan selected in the invention has a degree of deacetylation (DD) greater than or equal to 75%, preferably greater than or equal to 78%. Chitosan has the advantage of being one of the few natural cationic polysaccharides, which is easily usable and moreover has good adhesive properties.

The anionic polysaccharides are polymers of the carbohydrate family made up of several monosaccharides bound together by glycoside bonds. According to the invention, the polysaccharide or a salt thereof has a molecular weight between 500 and 1000 kDa. Among the anionic polysaccharides, we may mention for example glycosaminoglycans (GAGs), fucoidan, alginates, carrageenans, and ulvans.

The glycosaminoglycans (GAGs) are long-chain linear polysaccharides present in almost all tissues. The base unit of the GAGs is a disaccharide, consisting of a hexose (generally hexuronic acid) bound to a hexosamine One of the characteristic features of these oligosaccharide chains is their very considerable heterogeneity. In fact, the variable length of the chains and their structural modifications (sulfations, epimerizations) lead to countless combinations. Depending on the nature of the monosaccharides and the manner in which the disaccharides are joined together, the GAGs are classified in 5 main families the heparins (Hp) and the heparan sulfates (HS), hyaluronic acid (HA), the chondroitin sulfates (CS), the dermatan sulfates and the keratan sulfates.

Fucoidan is a polysaccharide having fucose as the base sugar. It takes its name from the algae of the fucus type (brown algae) in which it occurs.

The alginates are polysaccharides obtained from brown algae. The alginates are polymers formed from two monomers bound together by a β-1-4 linkage, namely mannuronic acid and guluronic acid.

The carrageenans are polysaccharides (galactan) extracted from red algae. Three main categories are marketed today: κ-carrageenan, l-carrageenan, and λ-carrageenan, which differ by the number and the position of the sulfate groups, as well as by the number of 3,6-anhydrogalactose bridges.

The ulvans are sulfated anionic polysaccharides extracted from green algae of the ulva type. They are made up of sodium ulvanobiuronate 3-sulfate type A comprising 3-sulfate rhamnose bound to glucuronic acid by a linkage of type 1-4 and of sodium ulvanobiuronate 3-sulfate type B comprising 3-sulfate rhamnose bound to iduronic acid by a linkage of type 1-4.

According to a particular embodiment of the invention, the anionic polysaccharide or a salt thereof is selected from a glycosaminoglycan (GAG), a fucoidan, an alginate, a carrageenan, and an ulvan. More specifically, the glycosaminoglycan (GAG) or a salt thereof is selected from hyaluronic acid (HA), heparin (Hp), heparan sulfates (Hs), chondroitin sulfates (CS), dermatan sulfates (DS), and keratan sulfates (KS), preferably hyaluronic acid, and even more preferably sodium hyaluronate.

Preferably, the anionic polysaccharide as defined above is hydrolyzable by the salivary enzymes.

Manufacture of the Mucoadhesive Patch

The mucoadhesive patch according to the present invention may be produced by assembly of layers that are positively charged (chitosan) and negatively charged (anionic polysaccharide) by an automated dipping process. This method of layer by layer (LbL) deposition is familiar to a person skilled in the art and is used in particular in international application WO 2005/052035 for preparing multilayer films of crosslinked polyelectrolytes.

The multilayer membrane thus formed is then brought into contact with a protein or a polypeptide to supply a mucoadhesive patch loaded with the protein or polypeptide.

The invention therefore relates to a method of manufacturing a patch as described in the present application, comprising the steps consisting of:
i) forming a CH/anionic polysaccharide multilayer membrane by a method of layer by layer (LbL) deposition on a substrate;
ii) detaching the multilayer membrane from the substrate;
iii) bringing the multilayer membrane into contact with a protein or a polypeptide, whereby the multilayer membrane is loaded with the protein or polypeptide.

Step i) of the method consisting of forming a CH/anionic polysaccharide multilayer membrane is carried out employing a technology of layer by layer (LbL) deposition on a substrate. This automated technology uses a dipping robot, for example the DR-3 robot from the company Riegler & Kirstein GmbH. More specifically, the solutions of chitosan and of the anionic polysaccharide (polyelectrolytes) are prepared in a buffer solution, such as a sodium acetate buffer solution. The pH of the solution may be adjusted to about 5.5 with sodium hydroxide (NaOH) and acetic acid (CH₃COOH). Moreover, the substrate used as support in the dipping robot is prepared on an adhesive tape. A substrate commonly used is polypropylene. The substrate is then immersed successively in the solution of chitosan and of the anionic polysaccharide with at least one washing step, preferably two washing steps in a buffer solution of sodium acetate, water or buffered saline solution between pH 5 and 6, preferably of sodium acetate. The cycle comprising immersion of the substrate in a solution of chitosan, at least one washing step, preferably a single washing step, followed by immersion in a solution of anionic polysaccharide and at least one washing step, preferably a single washing step, allows formation of a CHI/anionic polysaccharide bilayer. This cycle is repeated as many times as necessary to obtain a desired number of bilayers. The immersion and washing times may vary. According to a particular embodiment, the immersion time of the substrate in the solutions of polyelectrolytes (CHI and anionic polysaccharide) is between 2 and 10 minutes, preferably between 2 and 8 minutes, and more advantageously 3 minutes (Short Cycle, SC) or 6 minutes (Long Cycle, LC). According to another particular embodiment, the washing time is between 1 and 10 minutes, preferably between 2 and 4 minutes, and more advantageously 2 minutes (Short Cycle, SC) or 4 minutes (Long Cycle, LC).

Step ii) of the method, consisting of detaching the multilayer membrane from the substrate, in particular from polystyrene, may be carried out with an optional preliminary drying step. The multilayer membrane obtained may then be cut to the required size.

Carrying out step iii) of the method makes it possible to load the multilayer membrane with protein or polypeptide. More precisely, loading of the protein or polypeptide in the multilayer membrane is achieved by passive diffusion. In particular, step iii) is carried out by dissolving the protein or polypeptide in a suitable buffer solution so as to prevent denaturation of the protein. According to a particular embodiment, the multilayer membrane is brought into contact with a protein or polypeptide in an acid solution, preferably of hydrochloric acid (HCl), at a pH between 2 and 4, preferably at a pH of about 3. According to another particular embodiment, the multilayer membrane is brought into contact with a protein or polypeptide in a saline solution, preferably a solution of sodium chloride (NaCl) or a solution of potassium chloride (KCl), at a pH between 5 and 7, preferably at a pH of about 6.5.

Preferably, the contacting of the multilayer membrane with a protein or a polypeptide is carried out by depositing at least one drop of the buffer solution comprising the protein or the polypeptide on the surface of the multilayer membrane. The membrane is then optionally dried to supply a mucoadhesive patch in which the protein or the polypeptide is incorporated in the patch and/or adsorbed on the surface of the patch.

Preferably, and in particular in the case when the protein or the polypeptide remains at least partially adsorbed on the surface, the resultant patch is polarized, in that the concentration of protein or polypeptide is greater at the level of the upper layers where the protein or the polypeptide has been deposited, relative to the concentration at the level of the lower layers.

The method may also comprise an optional step of equilibration of the multilayer membrane before it is brought into contact with the protein or the polypeptide (step designated iii-0). This equilibration step consists of immersing the multilayer membrane obtained after step ii) in an acid solution, preferably of hydrochloric acid (HCl), at a pH between 2 and 4, or a saline solution, preferably of sodium chloride (NaCl), at a pH between 5 and 7. This optional step of equilibration of the multilayer membrane at acid pH allows in particular swelling of the membrane and thus facilitates loading with protein or polypeptide. The multilayer membrane loaded with protein or polypeptide may optionally shrink when it is soaked in a buffer solution, thus allowing trapping of the protein or polypeptide in the patch.

According to a particular embodiment, the method of the invention therefore further comprises an intermediate step consisting of equilibration of the multilayer membrane detached according to step ii) with a solution of hydrochloric acid (HCl) at a pH between 2 and 4, preferably at a pH of about 3 or of sodium chloride (NaCl) at a pH between 5 and 7, preferably at a pH of about 6.5, before step iii) is carried out.

A preferred method of manufacture according to the invention comprises the steps consisting of:
i) forming a CH/anionic polysaccharide multilayer membrane by a method of layer by layer (LbL) deposition on a substrate;
ii) detaching the multilayer membrane from the substrate;
iii-0) equilibration of the multilayer membrane detached according to step ii) with an acid solution, preferably of hydrochloric acid (HCl), at a pH between 2 and 4, preferably at a pH of about 3, or with a saline solution, preferably of sodium chloride (NaCl) at a pH between 5 and 7, preferably at a pH of about 6.5, before step iii) is carried out.
iii) bringing the multilayer membrane into contact with a protein or a polypeptide, whereby the multilayer membrane is loaded with the protein or polypeptide.

Loading the Patch

The protein or polypeptide may be incorporated in the patch or adsorbed on its surface, as described above.

Typically, the proteins or polypeptides of molecular weight below 70 kDa or 80 kDa are preferably incorporated entirely or practically entirely, i.e. preferably with at least 90% of proteins incorporated, in the patch. A protein or a polypeptide of higher molecular weight may be adsorbed wholly or partly on the surface of the patch.

The amount of protein or polypeptide incorporated and/or adsorbed depends on the protein or polypeptide and the desired biological or pharmacological effect. This amount may vary for example from 50 ng/cm² to 5 mg/cm², preferably from 10 ng/cm² to 1 mg/cm², 1 μg/cm² to 1 mg/cm².

Applications

The patch so obtained may be stored at 4° C. or at room temperature, before application.

Typically, the patch measures 2-3 cm² for application in a human subject. The patch may be applied for example on the inside of the cheeks, on the palate, the gum or under the tongue. A sublingual application (i.e. on the ventral face of the tongue) is particularly advantageous. Preferably, the patch is applied in such a way that the layer of chitosan on which the protein or the polypeptide was deposited during manufacture of the patch is the layer that is brought into contact with the mucosa.

The figures and examples illustrate the invention without limiting its scope.

### Example 1: Manufacture and Testing of a Mucoadhesive Patch

I. Materials and Methods

1.1 Materials

Chitosan (CHI) of medium molecular weight was purchased from Sigma-Aldrich.

Prior to use, the CHI was purified by steps of filtration and precipitation in water and ethanol, followed by lyophilization to obtain a final molecular weight of 770 kDa and a degree of deacetylation (DD) of 78%. At low pH (<6.5), CHI is a positively charged polyelectrolyte. It was compared with another polysaccharide, Viscosan® (VIS), obtained from Flexichem, which has a different distribution of the N-acetylated groups. The two types of sodium hyaluronate (HyA), of 610 kDa (HyA_{LW}) and 1020 kDa (HyA_{HW}), were purchased from HTL, France and were used as received. All the other reagents and solvents were used without purification.

For all the experiments, the solutions of polyelectrolytes were prepared extemporaneously by dissolution in a sodium acetate buffer (0.1 M CH₃COOH; 0.15 M NaCl, pH 5.5, at room temperature) using polymer concentrations of 1 mg/mL for production of the membranes.

1.2 Fluorescent Chitosan mL of medium-weight chitosan (CHI, 770 kDa, 10 mg·mL⁻¹ in acetic acid 0.1 mol·L⁻¹) was brought into contact to react with 20 mL of fluorescein isothiocyanate (FITC, 1 mg·mL⁻¹ in dehydrated methanol), for 3 h at room temperature and away from the light. The pH was then increased to 10 to precipitate the CHI, followed by centrifugations of 15 minutes at 11000 g and several washings with water until no fluorescence was detected in the supernatant. The CHI was then dissolved in 20 mL of acetic acid 0.1 mol·L⁻¹ and the unbound residue of FITC was removed by dialysis (Spectrum, USA) carried out in water for 3 days in the dark, the water being replaced daily. The quantities of CHI and FITC were determined by spectrophotometry at 490 nm and 270 nm respectively. The medium-weight CHI labeled with fluorescein (CHI^{FITC}) was divided into aliquots at 2 mg·mL⁻¹ in acetic acid 0.1 mol·L⁻¹ and stored at −20° C.

1.3 Manufacture of the CHI/HyA Membranes

The self-supporting membranes (CHI/HyA) were produced by the layer by layer (LbL) methodology using a dipping robot (DR-3, Riegler & Kirstein GmbH). The membranes were manufactured using polystyrene substrates cleaned by sonication in ethanol and distilled water (5 minutes for each solution). The substrates were immersed sequentially in CHI or Viscosan® and in solutions of HyA (HyA_{LW} of 610 kDa or HyA_{HW} of 1020 kDa) concentrated to 0.2% (weight/volume) in a sodium acetate buffer (CH₃COONa 0.2 M, CH₃COOH 0.2 M, pH 5.5, at room temperature), with 1 washing step using a sodium acetate buffer between each deposition in a solution of polymers. These immersions were repeated 100 times with a deposition time of 3 minutes for the polymers of natural origin and 2 minutes for each washing step. Then the membranes were left to dry at room temperature. Finally, the membranes were easily detached from their respective underlying substrate, simply by pulling them off using tweezers. The fluorescent membranes (CHI^{FITC}/HyA)₁₀₀-CHI^{FITC}) were prepared as described above, with 0.5% of CHI^{FITC} in the chitosan solution at 2 mg·mL⁻¹ in a sodium acetate buffer of pH 5.5, away from the light.

1.4 Thickness of the LbL Membranes

The thickness of the membranes produced was measured after drying and detachment of the substrate. The membrane thickness was determined using a micrometer (High-Accuracy Digimatic Micrometer, Mitutoyo); 20 measurements were carried out at different places at the level of the center of the membranes.

1.5 Artificial Saliva and Studies of Enzymatic Degradation

Membranes of 1 cm² were weighed before the experiment. An artificial saliva was prepared with α-amylase, hyaluronidase and lysozyme, dissolved in a buffer (0.15 M NaCl, mM HEPES, pH 6.5). All the enzymes have a final concentration of 100 μg·mL⁻¹. The samples were soaked in the artificial saliva and incubated at 37° C., stirring slowly for 30 min, 1 h, 3 h, 6 h or 24 h. After each period of incubation, the membranes were dried at 37° C. and weighed. The percentage weight loss (WL) of the membranes in the different conditions was determined from Equation 1, with Wi representing the initial dry weight of the membrane and Wf representing the weight of the dry membrane after each predetermined time point. Three independent experiments were carried out in triplicate for each condition and the mean value was taken as the percentage weight loss.

WL=(Wi−Wf)/Wi×100 (Equation 1)

For observation of the membranes in confocal laser scanning microscopy (CLSM), the fluorescent membranes were immobilized on glass plates and incubated in artificial saliva at 37° C. with stirring for 30 min, 3 h, 6 h or 24 h. Degradation was stopped by rinsing with an acetate buffer until observation in CLSM with an LSM710 confocal microscope (Carl Zeiss SAS, France). All the images were analyzed using Carl Zeiss Zen software and Image J software.

1.6 Scanning Electron Microscopy (SEM)

Morphological analysis of the samples (before and after degradation) was carried out using a scanning electron microscope (Merlin Compact VP, Zeiss) at an accelerating voltage of 5 kV. Both sides of the membranes were observed. Before observation, all the specimens were coated with copper (Balzers MED 010).

1.7 Cytotoxicity Tests

Immortalized Ho-1u-1 cells (a human cell line derived from squamous carcinoma cells from the floor of the mouth, obtained from GIMAP, St Etienne, France) were cultured in Dulbecco modified Eagle medium (DMEM) with D-glucose (4.5 g·L⁻¹), pyruvate (1 mmol·L⁻¹) and L-glutamine (2 mmol·L⁻¹), a DMEM/Ham's F12 cocktail of nutrients (1:1) supplemented with 10% (volume/volume) of heat-inactivated fetal bovine serum (FBS) and 1% (volume/volume) of penicillin/streptomycin. HeLa cells (human epithelial cell line derived from adenocarcinoma, ATCC® CCL-2) were cultured in DMEM with D-glucose (4.5 g·L⁻¹), pyruvate (1 mmol·L⁻¹) and L-glutamine (2 mmol·L⁻¹) containing 10% (volume/volume) of heat-inactivated FBS and 1% (volume/volume) of penicillin/streptomycin. The cells were kept at 37° C. in a 5% CO₂ atmosphere.

Two days before the cytotoxicity tests, the cells were seeded in 96-well culture plates. In parallel, the (CHI/HyA)100-CHI membranes were cut up to be resized (3 cm² per mL of medium), sterilized in 70% ethanol and by exposure to UV light. The membranes were then incubated overnight at 37° C. in a culture medium containing salivary enzymes (lysozyme, a-amylase and hyaluronidase) at 100 μg·mL⁻¹. The culture medium was then removed from the wells and replaced with the medium containing the degradation products. The cells were incubated at 37° C. for 24 h. Then methylthiazolyldiphenyl-tetrazolium bromide (MTT, mg·mL⁻¹) was added to each well for 3 h incubation at 37° C. The cells were then incubated overnight, at 37° C. and away from the light, in a solubilizing solution containing 10% (volume/volume) of Triton X-100 and HCl (0.1 mol·L⁻¹) in anhydrous isopropanol. The absorbance was measured at 570 nm and 690 nm (i-control Infinite® M1000 Pro, Tecan, Switzerland). Positive controls were carried out with 0.1% (volume/volume) of sodium dodecyl sulfate (SDS) and negative controls with cells only. The data were taken as the mean value of triplicates for three independent experiments.

1.8 Mice

Studies in vivo were carried out on female CB6F1 mice aged from 6 to 8 weeks (Charles River Laboratories, France) and on male SHK-1 mice (Charles River Laboratories, France) for tomography experiments. All the animals were kept in pathogen-free conditions. All the animal studies were carried out in compliance with the directives of the European Union and approved by the regional and national ethics committees.

1.9 Sublingual Administration of Membranes or Solutions

The membranes were cut up to be adjusted to the size of the mouse tongues (2 mm×7 mm) and sterilized by UV light. The membranes or liquid formulas were then administered by the sublingual route (ventral part of the tongue) to lightly anesthetized mice (isoflurane 4%). After administration, light pressure was applied for 10 s (until awake) on the dorsal part of the tongue to ensure contact of the membrane or liquid solution with the mucosa. No other restraint was carried out. After recovery from the anesthesia, the animals were left free to drink or to groom. Water was withdrawn for the 30 min following administration and was returned for longer experiments.

1.10 Histologic Analysis of the Swelling of the Mucosae and Recruitment of the MHCII Positive Cells

Native membranes, membranes treated with HCl or membranes treated with NaCl were administered for 30 min or 60 min. A volume of 12.5 μl of a solution of 1-fluoro-2,4-dinitrobenzene (DNFB) at 0.5% was used as positive control for inflammation. The negative control group did not undergo any manipulation. Each group comprised 3 mice. For the entire treatment time, the mice were alert, and water was removed for the first 30 minutes. At each time point, the tongues were placed in an OCT® matrix (Optimal Cutting Temperature, Tissue Tek) and were stored at −80° C. until cryosection. For the cryosection step, slices with a thickness of 6 μm were prepared using a cryostat (Leica) and fixed on glass plates with acetone at −20° C. For analysis of the swelling of the mucosa, the slices of tongue were stained with hematoxylin (Gill formula, Vector) and the images were captured using an inverted microscope (Nikon Ti-E). The swelling of the mucosae was analyzed on lengths of 2 mm extending from the base of the ventral surface. Measurements of area were carried out using the polygon tool of the Image J software. For staining of the MHCII, the slices of tongue were first incubated with a peroxidase blocking reagent (Dako), then with biotinylated rat antimouse antibodies (BD pharmigen), developed using the Vectastain Elite ABC kit (Vector) and the AEC peroxidase substrate (Vector), and finally counterlabeled with hematoxylin (Gill Formula, Vector). The slices were examined using a Nikon Ti-E microscope. The number of MHCII positive cells (MHCII+) was counted on lengths of 2 mm extending from the base of the ventral surface of the sublingual membrane, using the plug-in Cell Counter of the Image J software.

1.11. Preparation of the Specimens for Quantification of Cytokines

Each group comprised 3 mice. Two groups received native membranes or membranes treated with HCl. Three other groups of mice received, by the sublingual route, 10 μL of CHI (770 kDa, 2 mg·mL⁻¹ in acetate buffer pH 5.5, Sigma, USA), 10 μL of HyA (610 kDa, 0.95 cm³·kg⁻¹, 2 mg·mL⁻¹, in acetate buffer pH 5.5, HTL, France), or 10 μL of a combination of the two polymers. The control groups of mice received, by the sublingual route, either of PBS or of acetate buffer pH 5.5 for the negative controls, or 12.5 μL of DNFB at (volume/volume) in chloroform for the positive controls. The tongues were excized 6 h after application of the membranes or solutions, frozen in liquid nitrogen and stored at −80° C. Briefly, the tongues were incubated in RIPA buffer [Tris HCl (50 mmol·L⁻¹), NaCl (150 mmol·L⁻¹), Triton X-100 (1%), sodium deoxycholate (0.5%), SDS (0.1%), EDTA (1 mmol·L⁻¹) and a protease inhibitor cocktail (1%, Thermo Scientific)] for 2 h on ice, after homogenization using scissors. The preparations were then homogenized using a ball-type beater (2×5 min, 30 Hz, 4° C.) (TissueLyzer II, Qiagen, Germany) and sonication (2 min, 60 Hz), followed by centrifugation for 10 min at 10 000 rpm and 4° C. The total protein concentration in the supernatants was then determined using the BCA protein assay kit (ThermoFisher Scientific, USA). Interleukin 1 beta (IL-1β), IL-6 and tumor necrosis factor alpha (TNF-α) were quantified simultaneously in each sample (V-Plex Proinflammatory Panel 1 Mouse Kit, MSD, USA) by electroluminescence using the Mesoscale Discovery system (Meso QuickPlex SQ 120, MSD, USA). The data were obtained as the mean value of duplicates with three mice for each condition.

1.12 Incorporation of Proteins by Swelling as a Function of pH

For the purpose of evaluating the capacity of the membranes for incorporating proteins at different values of pH, the membranes were equilibrated with 1 mM of HCl (pH 3-3.5), a phosphate-buffered saline solution (PBS 1×, Gibco® by Life Technologies™ pH7.4) or with a sodium chloride buffer (NaCl 0.15 mol·L⁻¹, HEPES 0.02 mol·L⁻¹, pH 6.5) at room temperature for 1 h. Before the experiments, membranes with a diameter of 12 mm were sterilized by exposure to UV for 20 mM. After equilibration of the excess HCl, the PBS or the NaCl buffer was removed and a drop of protein was deposited on top of the membrane. The protein used was either ovalbumin labeled with Alexa Fluor 647 (OVA^{AF647}) at 5 μg·mL⁻¹ (500 ng loaded) or cytokine CCL20 at 500 ng·mL⁻¹ (50 ng loaded) in 1 mM of HCl or NaCl buffer. The membranes were incubated overnight at 4° C. After rinsing with acetate buffer pH 5.5 and drying under an air stream, the functionalized membranes are designated “bioactive patch”.

1.13 Study of Ovalbumin Release

Before the experiments, OVA^{AF647} was incorporated in the membranes as described above. The release of the proteins was monitored at varying pH, acetate buffer at pH 5.5, PBS at pH 7.4 of Dulbecco (dPBS) or artificial saliva at pH 6.5 being used as rinsing solutions. Immediately after adding the rinsing solutions, the proteins were removed and stored for evaluating the amount of unbound proteins (“quick rinse”, QR). Release of OVA^{AF647} from the membrane was carried out for 2 h with time points at 10, 20, 30, 40, 60, 90 and 120 min. The OVA^{AF647} released was analyzed by fluorescence spectroscopy (Infinite M1000, Tecan) with the excitation/emission wavelength of Alexa Fluor 647 fixed at 650/668 nm. A calibration curve for the labeled protein was recorded in dPBS, acetate buffer, HCl and the artificial saliva at the aforementioned wavelengths.

1.14 Tomographic Analysis of Muco-Adhesion of the Patch and of the Protein Retention Time

For the tomographic analyses of the protein retention time in the buccal region of the mice, groups of 2 mice were anesthetized in a chamber receiving a flow of 4% isoflurane for minutes. For administration of the solution, 10 μl of solution of OVA^{AF647} was deposited at the base of the ventral face of the mouse tongues. Regarding administration of the patches, the latter were placed on the ventral surface of the tongues. For each formulation, 5 μg of OVA^{AF647} was administered. The mice were placed in the tomographic chamber (FMT 4000, Perkin Elmer) under isoflurane, positioned lying head first for image acquisition 2, 10 or 30 minutes after administration.

1.15 Penetration of Ovalbumin in the Sublingual Mucosa

The penetration of OVA^{AF647} in the sublingual mucosa was evaluated after administration of a liquid formulation (10 μl) or a (CHIFITC-HyA)100-CHI patch. OVA^{AF647} was incorporated by incubation in HCl, as described in 1.12. Groups of 2 mice received the various formulations and were euthanized 2, 10, 30 or 60 minutes after administration. The sublingual mucosae (ventral face of the tongue and floor of the mouth) were taken, embedded in an OCT® matrix and stored at −80° C. Sections of 40 μm were prepared, labeled with the DAPI nuclear probe and then observed with the confocal microscope (LSM 710, Zeiss, Germany)

1.16 Chemotaxis Test

A mouse dendritic cell line (DC 2.4, #SCC142 Millipore) was used for in vitro testing of the chemotaxis effect of chemokine CCL20 delivered by the membranes that have been developed. Cells were cultured in RPMI medium supplemented with GlutaMAX, with 10% of FBS, 10 mM of HEPES, 50 μM of 0-mercaptoethanol and a mixture of nonessential amino acids (1×) (called GM hereinafter) at 37° C. and under an atmosphere with 5% CO₂. The chemotaxis test was carried out in inserts of cell culture ThinCert™ (Greiner Bio-One; ref: 665 610) placed in 12-well plates, for 20 minutes. Before the test was carried out, the membranes, chemokine CCL20 and the membranes loaded with chemokine CCL20 (500 ng·mL⁻¹) were incubated for 24 h in a solution of salivary enzymes (0.1 mg·mL⁻¹ of α-amylase, of hyaluronidase and of lysozyme dissolved in GM without FBS) at 37° C., stirring slowly. Then the inserts of the 12-well plate were seeded at a density of 2.0×10⁵ cells/insert and incubated for 10 minutes at 37° C. Then the lower chambers of the 12-well plate were carefully filled with 1 mL of chemotactic solution or of control solution, for incubation for 10 minutes at 37° C. The positive control corresponded to dendritic cells (DC) incubated in the presence of GM only. Finally, the internal faces of each insert were wiped using a cotton bud. The samples were observed with an inverted microscope (Nikon Ti-E) after fixation (with 4% paraformaldehyde) and staining with DAPI. The results obtained represent the mean value of the triplicates of 3 independent experiments.

1.17 Statistical Analysis

All the data were analyzed and entered in version 7.0 of the Graphpad Prism software. The quantities indicated represent the mean value±standard deviation (SD) of at least three replicates; apart from the release data representing the mean value±standard error (SEM). p<0.05 was considered statistically significant after an ANOVA test with a single controlled factor, with the Tukey multiple comparison test or with the Dunnett multiple comparison test for the cytotoxicity tests.

2. Results

2.1 Degradation of Self-Supporting Membranes in Artificial Saliva

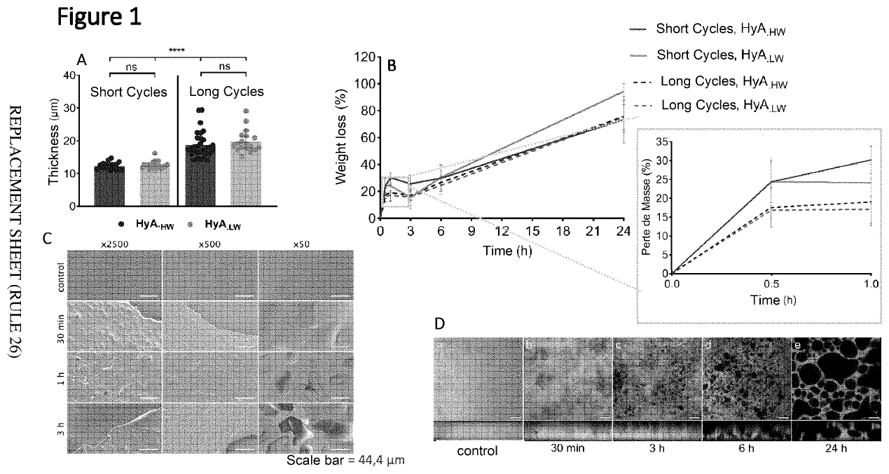

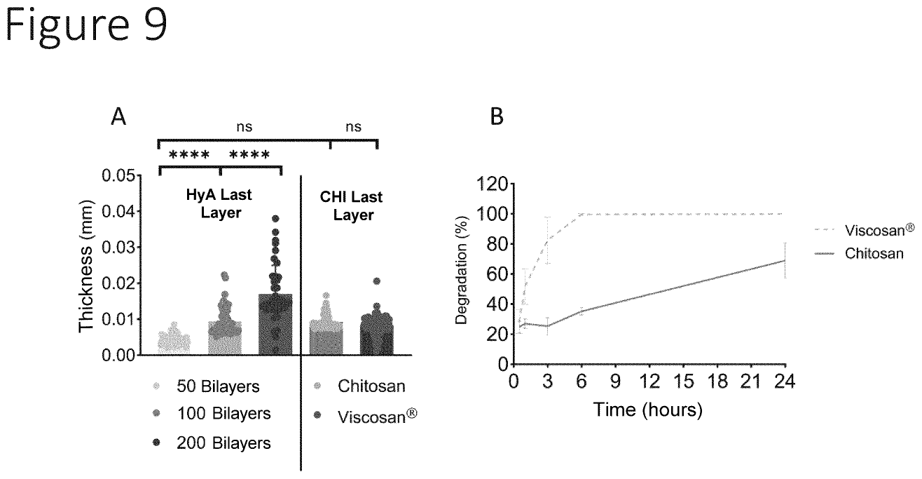

The (CHI/HyA)₁₀₀ native membranes were produced by varying 3 parameters: the molecular weight (MW) of HyA, the deposition time of the polyelectrolytes and of the rinsing solutions, and the properties of the CHI. The influence of these parameters on membrane thickness was examined first. It was shown that the growth of the (CHI/HyA)₁₀₀ native membranes is linear with membranes of 50 bilayers (4.5±1.39 μm), of 100 bilayers (10.30±7.67 μm) and of 200 bilayers (17.04±7.96 μm) (FIG. 9). At least 50 bilayers were necessary for easy manipulation without any post-procedure treatment. No significant difference was found at the level of the membrane thickness by varying the MW of HyA (610 KDa or 1000 kDa) (FIG. 1A). Regarding the deposition time, namely the long cycles (LC, 6 min for the polyelectrolytes and 4 min for the rinsing buffer) and the short cycles (SC, 3 min for the polyelectrolytes and 2 min for the rinsing buffer), significant differences were observed at the level of the membrane thickness, where an increase of about 32% was found when the membranes were produced under LC conditions (FIG. 1A).

The influence of the conditions used for construction of the membranes on the degradation was also examined. Moreover, as the intention is to use the membranes for sublingual applications, an artificial saliva was produced. The artificial saliva consisted of a physiological solution containing α-amylase, lysozyme and hyaluronidase, three enzymes that occur in human saliva. The degradation of the membranes produced was monitored over a period of 24 h and quantified, being expressed as percentage weight loss. The membranes produced with Viscosan® degraded more quickly than the membranes based on CHI (FIG. 9B), as expected owing to the rapid biodegradability of Viscosan®. Although the general profile of degradation of the membranes based on CHI was not affected drastically by the molecular weight of HyA, nor the deposition times (FIG. 1B), in the initial phase of the degradation process (in the first hour) a delay was found with the membranes constructed in LC conditions (insert in FIG. 1B). After 24 h, all the membranes have reached a level of weight loss in the range from 75% to 95%.

In order to obtain morphological information concerning their degradation, the (CHI/HyA_{LW})₁₀₀ membranes were observed in SEM and in confocal microscopy. On observing the images from these two techniques, an initial surface erosion was observed at 30 min, followed by the formation of surface holes at 1 h, and finally of deeper holes at 3 h (FIGS. 1C and 1D). The enzymatic degradation of the surface of the membrane was also observed starting from 30 min of immersion in artificial saliva, in hydrated conditions where the membrane has swollen to about 30 μm (FIG. 1D). No degradation was observed after 24 h in the acetate buffer (FIG. 1D, control).

Because these membranes have been designed for administration of the protein via the mucosae, the inventors selected membranes made of CHI instead of Viscosan® to ensure prolonged diffusion of the cargo protein from the first stages of degradation in contact with the mucosa. The membranes produced in SC conditions were also selected to reduce the production time, and the HyA_{LW} of 610 kDa was selected at random as no difference in thickness or degradation was observed relative to HyA_{HW}. To ensure optimal mucoadhesion, all the membranes used for the animal experiments had a first and last layer of CHI. Thus, for the next studies, the (CHI/HyA_{LW})₁₀₀-CHI membranes were simply designated with (CHI/HyA)₁₀₀.

2.2 Cytotoxicity Toward Human Epithelial Cells

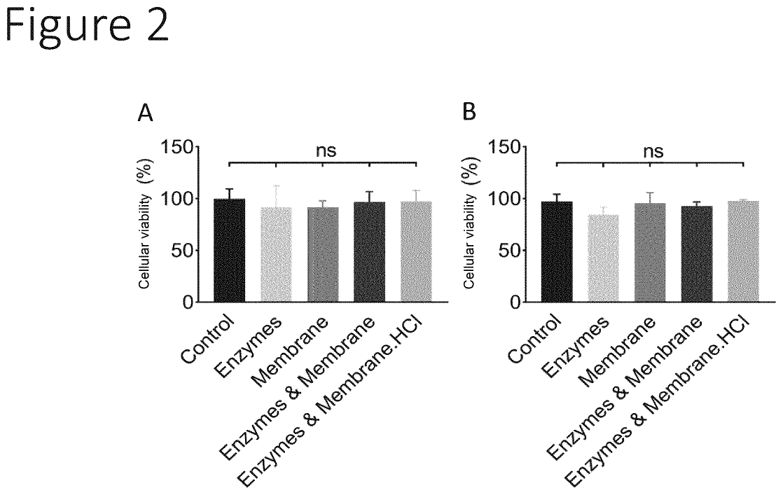

The cytotoxicity of the degradation products of the membranes was evaluated on two human epithelial cell lines (HeLa and Ho-1u-1) over an incubation time of 24 h. No toxicity was observed on the two cell lines as the viability remained around 100% (FIG. 2A-B). This result is explained by the known biocompatibility of the two polysaccharides used for producing the membranes, which had not been modified chemically. Combining the polymers did not affect their biological safety. Consequently, the membranes may be applied in vivo on the sublingual mucosae of mice, without leading to a cytotoxic effect on the epithelial cells of the tissue.

2.3 Inflammation of the Mouse Sublingual Mucosa

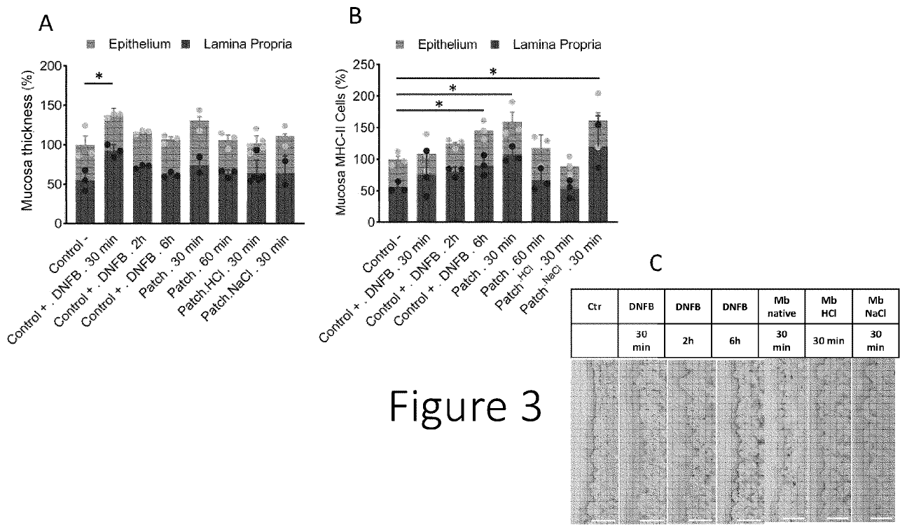

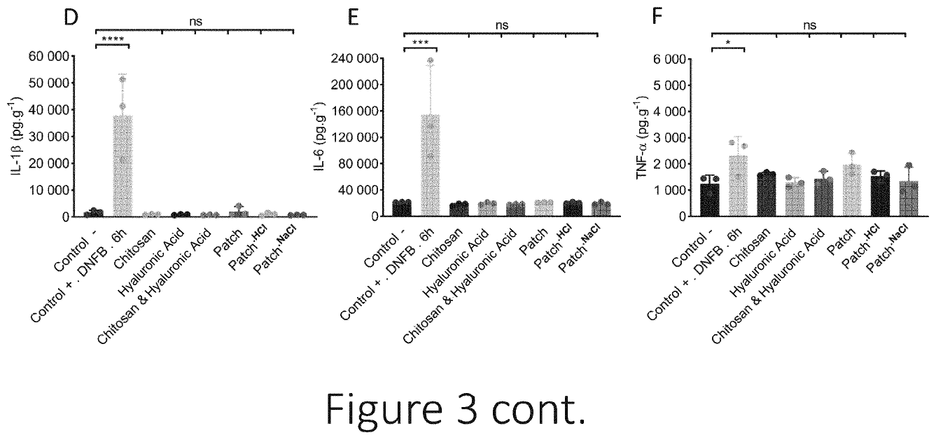

The inflammatory response induced in vivo by the patch was evaluated on mice by evaluating various principal characteristics of the inflammatory state. First, the swelling surface of the epithelium and lamina propria of the sublingual mucosa of the mice was observed in different conditions. As the positive control of inflammation, 1-fluoro-2,4-dinitrobenzene (DNFB) at 0.5% was used as it had been demonstrated that it induces inflammation when it is administered by the sublingual route [LeBorgne et al., 2006]. An increase in the thickness of the mucosa was observed 30 min after administration of DNFB and decreased slightly after 2 h (FIG. 3A). It should be noted that the swelling of the lamina propria was maintained for 6 h whereas the thickness of the epithelium had already decreased after 2 h. In order to quantify the swelling induced by the (CHI/HyA)₁₀₀ membrane, native membranes and membranes treated with HCl (incubation for 1 h in 1 mM of HCl, pH 3) were applied on the sublingual mucosa for 30 or 60 mM. As the treatment with HCl forms part of the protocol for incorporation of the proteins, its effect on the inflammatory response was evaluated. The swelling of the mucosa after application of the native membrane was comparable to the swelling induced by the DNFB positive control at 2 h, whereas the mucosa in contact with the membrane treated with HCl was significantly thinner. In order to correlate the swelling of the tissues with the infiltration of the immune cells, immunohistochemical staining of the major histocompatibility complex of class II (MHCII) was carried out. MHCII is a major marker of the antigen-presenting cells (APC), in particular of the dendritic cells (DC), B lymphocytes and macrophages. The degree of infiltration of the MHCII+ cells in the mucosa had increased significantly 30 minutes after administration of the native membrane (FIG. 3B-C) and had returned to the level of the control after 60 minutes. It is interesting to note that the membranes treated with HCl applied for 30 mM did not cause infiltration of MHCII+ cells, the infiltration remaining similar to that of the control, as observed for the swelling of the mucosa (FIG. 3A).

To identify the profile of inflammation in the medium term induced by the native membranes and the membranes treated with HCl, the profiles of expression of the inflammatory cytokines were quantified 6 h after administration of the patch. In all the conditions tested (polymers in solution, or membranes with or without treatment with HCl), the levels of IL-1β, IL-6 and TNF-α were similar to the levels of the control, which means that no inflammatory signaling had been induced 6 h after application of the patch.

2.4 Incorporation/Release of a Model Protein, Ovalbumin

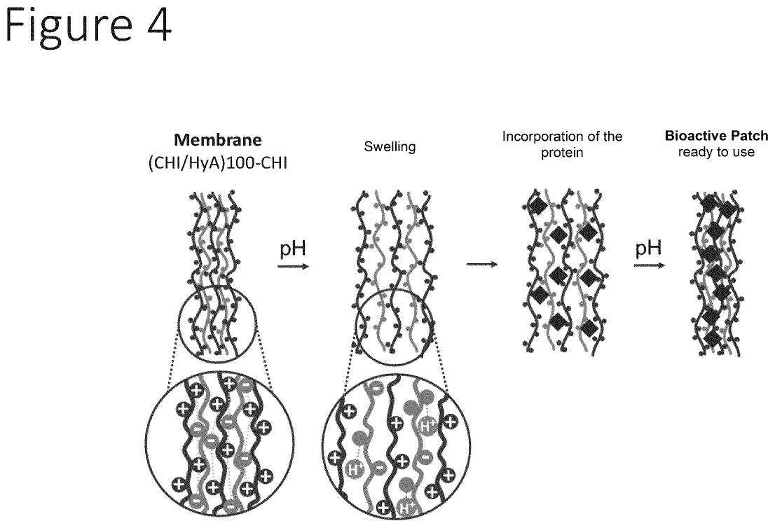

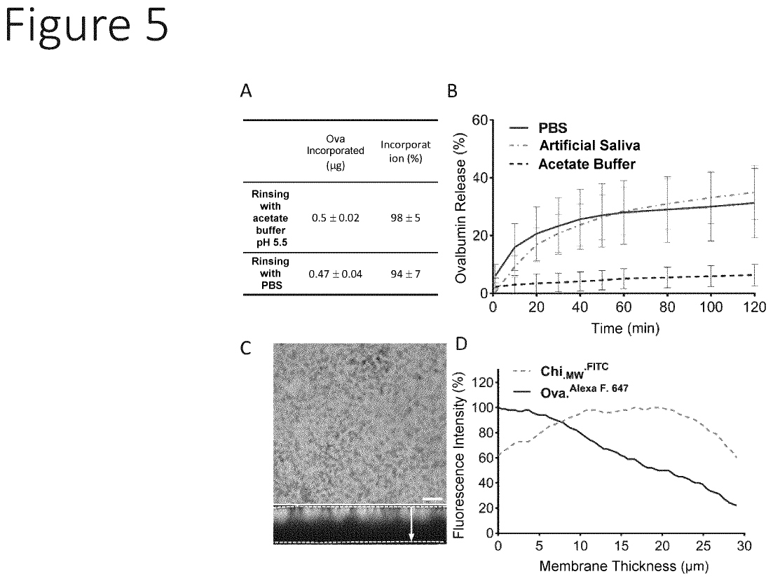

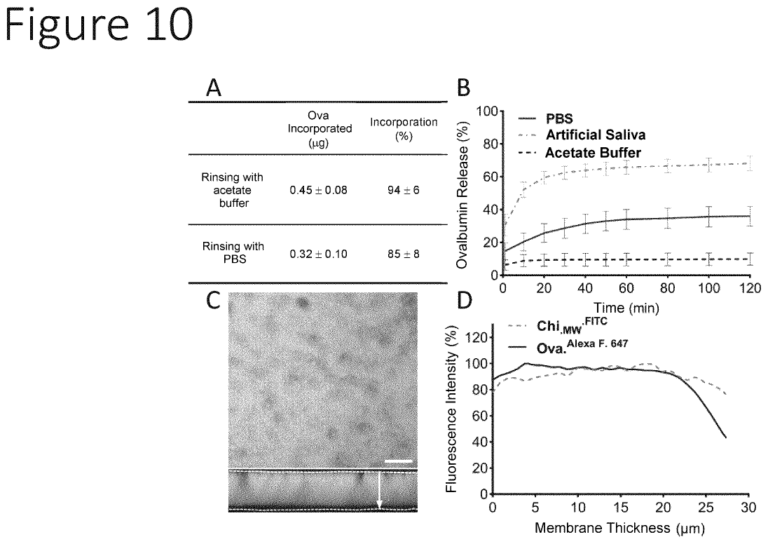

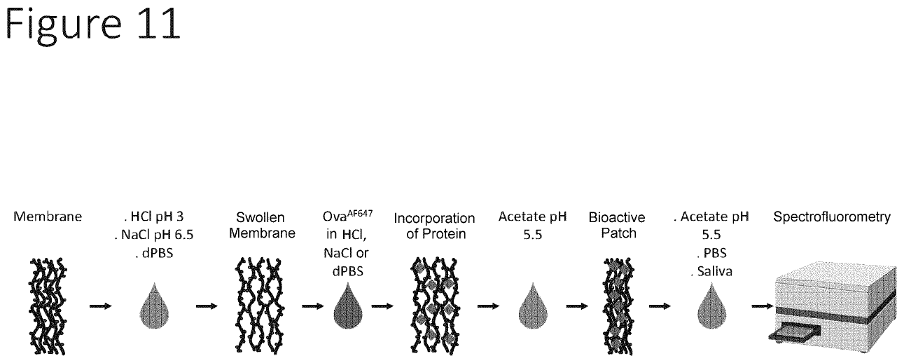

To evaluate the (CHI/HyA)₁₀₀ membranes as systems for diffusion of proteins, the latter were functionalized with a model protein, ovalbumin, labeled with the fluorophor Alexa Fluor 647 (OVA^{AF647}). OVA^{AF647} was incorporated in the membranes after equilibration of the membranes in HCl 1 mM, pH 3 (according to the scheme in FIG. 4). Rinsing of the membranes with an acetate buffer pH 5.5 (rinsing buffer for the superposition of the membranes) made it possible to incorporate 98% of the OVA^{AF647} whereas rinsing with PBS (pH 7.4) led to a slight decrease in incorporation (94%) (FIG. 5A). The kinetics of release over 2 h confirmed these differences since, in the acetate buffer, less than 7% of the OVA^{AF647} was released from the patch, against 31% in PBS (FIG. 5A-B). On immersion in the artificial saliva, 35% of the OVA^{AF647} was released from the patch in 2 h, and about 90% after 24 hours of immersion (data not presented). The profile of incorporation of OVA^{AF647} at a pH of 3 followed by rinsing with acetate buffer showed that about 80% of the protein was incorporated in the first 10 micrometers under the surface, which corresponds to one third of the total hydrated thickness (FIG. 5C-D). The functionalized membranes have thus become an asymmetric patch, the surface with the protein being the surface that is in contact with the mucosa. As certain proteins may be sensitive to acid pH (denaturation, loss of activity etc.), incorporation of OVA^{AF647} was also carried out at a physiological pH of 7.4 in PBS instead of HCl at pH 3. However, it was not possible to manipulate or visualize the patch after incorporation of the proteins, on account of the considerable swelling of the membrane. Furthermore, the instability of the structure led to a degree of incorporation below 60% (data not presented). Instead, an NaCl buffer (NaCl 0.15 mol·L⁻¹, HEPES 0.02 mol·L⁻¹, pH 6.5) was used for incorporation of the proteins. For rinsing the membranes, the same solutions were tested (acetate buffer, PBS and artificial saliva). As observed for loading at acid pH, rinsing with an acetate buffer led to 94% of incorporation of proteins whereas rinsing with PBS only led to 85% of incorporation of OVA^{AF647} (FIG. 10A). The release profiles confirmed that the acetate buffer ensures retention of the proteins inside the membrane, as less than about 10% of the OVA^{AF647} is released from the membrane, whereas PBS leads to a rapid release of about 36% of OVA^{AF647} (FIG. 10B). The release of the protein after 2 h of rinsing in the artificial saliva reached 69%. When it is incorporated with NaCl buffer, the distribution of the protein inside the patch proved more homogeneous than incorporation with HCl, and did not present a depthwise gradient of penetration, along the Z axis (FIG. 10C, D).

2.5 Mucoadhesion and Retention Time of the Cargo Protein

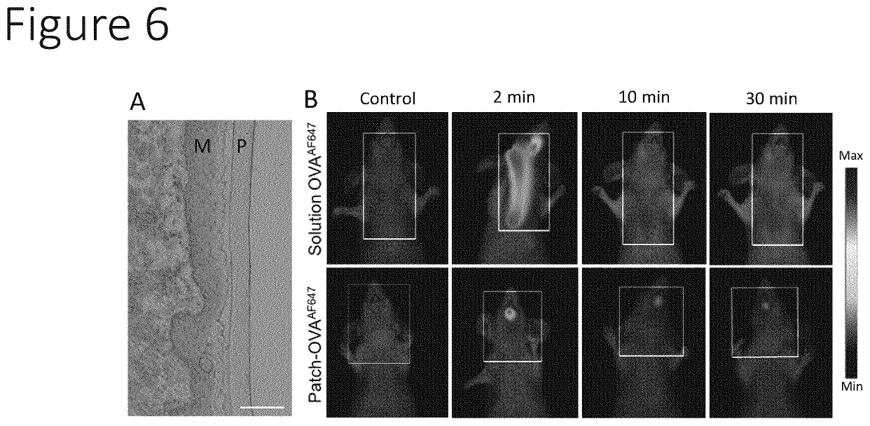

The mucoadhesion of the patches was evaluated in vivo by visualization of the membrane resting on the sublingual mucosa 20 min after administration (FIG. 6A). Monitoring of OVA^{AF647} by molecular fluorescence tomography demonstrated the rate of dispersion of the protein after administration as a liquid formulation (FIG. 6B). During the first minutes of image acquisition, OVA^{AF647} was already distributed along the alimentary canal and no signal was detectable in the mouth after 10 min. When it was co-administered with the mucoadhesive membrane, OVA^{AF647} was detected in the form of a concentrated signal in the mouth for at least 30 min.

2.6 Presentation on the Mucosa and Penetration of the Cargo Protein

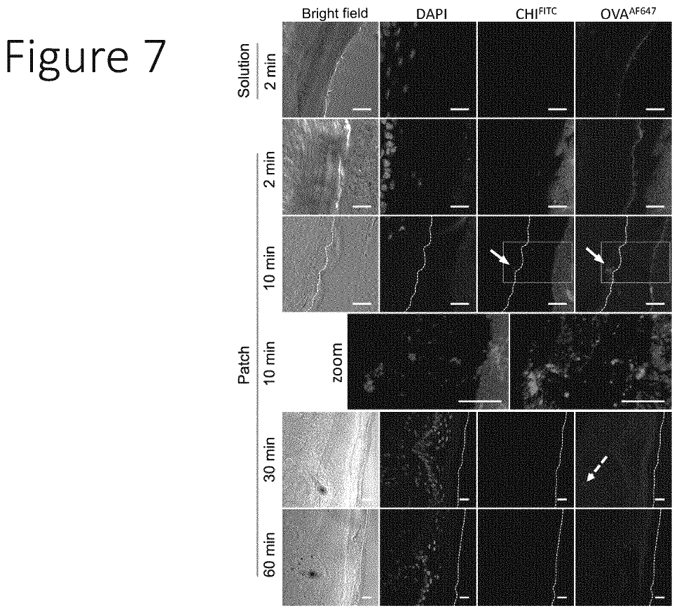

The tissue penetration of OVA^{AF647} was monitored by confocal microscopy. The intensity of the signal 2 min after administration was much weaker in the liquid form than that displayed by the membrane (FIG. 7). OVA^{AF647} had penetrated into the keratinized layer 10 min after administration of the patch as well as in the CHI^{FITC} (white arrows, FIG. 7). The patch was not detectable 30 minutes after application, but OVA^{AF647} was present deep in the mucosa (dashed white arrow, FIG. 7). Even if accumulation was detected in the epithelium (surface layer), OVA^{AF647} was observed in the lamina propria and the submucosa. The signal was barely detectable 60 minutes after administration. This decrease in the fluorescence signal of the protein 60 minutes after administration relative to 30 minutes might be explained by the clearance of the protein in the tissue and its absorption by immune cells such as the APCs.

2.7 Bioactivity of the Incorporated Protein

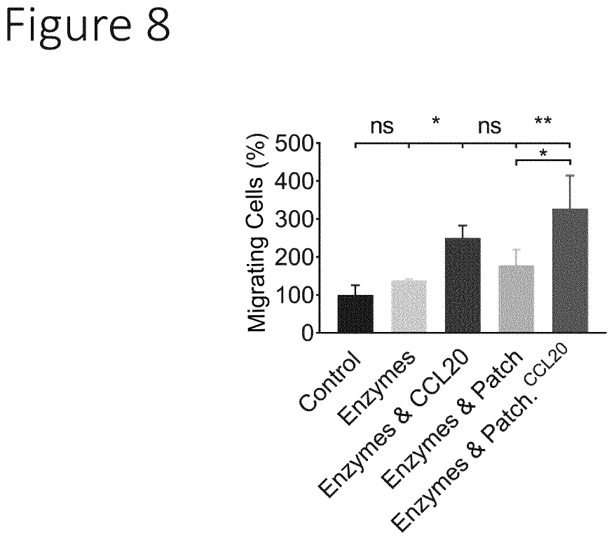

To evaluate the functionality and bioactivity of a protein incorporated in the (CHI/HyA)₁₀₀ membrane, a chemoattractive cytokine (CCL20) was loaded in the assembly and sampled after degradation of the membrane in the artificial saliva. Cytokine CCL20 was used as chemoattractant for the murine dendritic cells (DC 2.4) that possess the associated CCR6 receptor (data not presented). The chemoattractive capacities of the cytokine were preserved within the patch, as a similar migration was observed for CCL20 in solution and CCL20 collected after dissolution of the patch (FIG. 8). The bioactivity of the protein was therefore preserved by the patch and the cytokine displayed a significant chemoattractant effect on the targeted cells.

### Example 2: Evaluation of the Rate of Incorporation in (CHI/HyA)₁₀₀ Patches

1. Incorporation of Bovine Serum Albumin (BSA)

Materials and Methods

The (CHI/HyA)₁₀₀ membranes were cut up so as to obtain 1 cm² squares, which were incubated either in HCl buffer solution of pH 3, or in NaCl buffer solution of pH 6.5, both containing serum bovine albumin (BSA) at a concentration of 10 mg·mL⁻¹ The squares of (CHI/HyA)₁₀₀ membranes were covered completely with a volume of liquid of the order of 200 μl; at equilibrium, they each contain about 2 mg of absorbed BSA. The squares of (CHI/HyA)₁₀₀ membranes loaded with BSA were incubated in a release buffer (acetate buffer, pH 5.5) without BSA, in order to measure the amount of proteins released, using a protein assay kit based on bicinchoninic acid (BCA) for a time of 4 h. Measurements of optical density (562 nm) were performed by taking samples of the release buffer at the start (t0) and after 5, 10, 20, 30, 60, 120 and 240 min of incubation, the release buffer being replaced after each sampling. Measurement of a blank obtained with wells containing only the incubation solution was systematically deducted. From a standard curve, the concentration of proteins per ml could be determined for each release time, and then referred to an amount of BSA for 200 μl. Finally, the quantity of proteins incorporated in the membrane was found by subtracting the amount of BSA determined for 200 μl from the theoretical 2 mg of BSA absorbed initially.

Results

The amount of BSA incorporated per cm² in (CHI/HyA)₁₀₀ membranes was determined by assay of the BSA released from the membranes pre-incubated in a concentrated solution of BSA at different pH. Very similar release profiles were observed between the BSA incorporated at pH 3 and the BSA incorporated at pH 6.5, with respectively 0.79 mg and mg released instantaneously (t0), additional 0.18 mg and 0.16 mg released after 5 minutes, and again 0.03 mg and 0.01 mg released after 10 minutes. After 20 minutes of release, the BSA released between times t10 and t20 was no longer detectable, which indicates that release mainly occurs in 10 minutes. The measured total amount of BSA released corresponds to 1.01 mg for BSA incorporated at pH 3 and to 0.92 mg for BSA incorporated at pH 6.5. Consequently, the squares of (CHI/HyA)₁₀₀ membrane that initially absorbed about 2 mg of BSA still have 1 mg of BSA after 4 h of rinsing, or about 0.99 mg of BSA for incorporation carried out at pH 3 and about 1.08 mg for incorporation carried out at pH 6.5. The rate of incorporation is therefore 50% in both conditions.

2. Incorporation of Immunoglobulins (IgG) in the (CHI/HyA)₁₀₀ Membranes

Materials and Methods

(CHI/HyA)₁₀₀ membranes were cut up so as to obtain 1 cm² squares, which were incubated for 1 h either in 200 μl of HCl equilibration solution of pH 3 (samples MbA) or in 200 μl of NaCl-Hepes equilibration solution of pH 6.5 (samples MbB). The squares of MbA membrane were then incubated in 150 μl of an incorporation solution based on HCl of pH 3 containing 2 μg·mL⁻¹ of immunoglobulin of type G (IgG; donkey secondary antibodies directed against goat IgG and coupled to the fluorochrome Alexa 633; Invitrogen, Molecular probes A21082-lot 73A2-1), and the squares of MbB membrane were incubated in 150 μl of an incorporation solution based on NaCl-Hepes of pH 6.5 also containing 2 μg·mL⁻¹ IgG. In parallel, squares of dry (CHI/HyA)₁₀₀ membrane (MbC and MbD), which had not undergone the equilibration step, were incubated respectively in the same incorporation solutions as the samples MbA and MbB. The incubations in the incorporation solutions were carried out overnight, at 4° C. The next day, the membranes were rinsed 3 times for 2 minutes in acetate buffer (0.1M CH₃COOH, 0.1M CH₃COONa, pH 5.5) and then dried under an air flow (PSM) away from the light. The membranes were finally observed with the confocal fluorescence microscope (Zeiss, LSM 710) using a ×20 objective.

Results

The membranes (Alexa 633 nm) with or without a preliminary equilibration step at different pH were examined (images not supplied). The presence of IgG could be detected comparably and in all the conditions, essentially at the surface of the membranes MbA, MbB, MbC and MbD that was opposite the wall of the plate during the incubations. However, the labeling of the IgG is more pronounced for the MbA samples, which indicates that the preliminary equilibration step would offer an advantage only for incorporation at pH 3. Moreover, the amount of IgG incorporated in the membranes could be estimated at about 400 ng per cm².

### REFERENCES

Caridade et al., Biomacromolecules. 2013, 14(5): 1653-60
Caridade et al., Acta Biomater. 2015, 15:139-49
LeBorgne et al., Immunity. 24 (2006) 191-201

1. A buccal or sublingual mucoadhesive patch, intended for buccal or sublingual release of a protein or of a polypeptide, said patch comprising at least 100 bilayers each made up of a layer of chitosan (CHI) and a layer of an anionic polysaccharide having a molecular weight between 500 and 1000 kDa or a salt thereof, wherein the layer in contact with the mucosa and the layer in contact with the buccal or sublingual environment of the patch consist of chitosan (CHI), and wherein said protein or said polypeptide are incorporated in said patch and/or adsorbed on the surface of said patch.

2. The patch according to claim 1, comprising from 100 to 200 CHI/anionic polysaccharide bilayers.

3. The patch according to claim 1, having a thickness from 10 to 20 μm.

4. The patch according to claim 1, wherein the anionic polysaccharide or a salt thereof is selected from the group consisting of a glycosaminoglycan (GAG), a fucoidan, an alginate, a carrageenan, and an ulvan.

5. The patch according to claim 4, in wherein the glycosaminoglycan (GAG) or a salt thereof is selected from the group consisting of hyaluronic acid (HA), heparin (Hp), heparan sulfates (Hs), chondroitin sulfates (CS), dermatan sulfates (DS), and keratan sulfates (KS).

6. The patch according to claim 1, wherein the protein or polypeptide is an allergen.

7. A method for desensitizing a subject allergic to an allergen, the method comprising administering to the subject said allergen in the form of a patch, wherein the allergen is a protein or polypeptide; the patch is a buccal or sublingual mucoadhesive patch comprising at least 100 bilayers, each made up of a layer of chitosan (CHI) and a layer of an anionic polysaccharide having a molecular weight between 500 and 1000 kDa or a salt thereof; the layer in contact with the mucosa and the layer in contact with the buccal or sublingual environment of the patch consist of chitosan (CHI); and said allergen is incorporated in said patch and/or adsorbed on the surface of said patch.

8. A method of manufacturing a patch as defined in claim 1, comprising the steps of:
i) forming a CH/anionic polysaccharide multilayer membrane by a method of layer by layer (LbL) deposition on a substrate;
ii) detaching the multilayer membrane from the substrate; and
iii) bringing the multilayer membrane into contact with a protein or a polypeptide, whereby the multilayer membrane is loaded with the protein or polypeptide.

9. The method according to claim 8, wherein the multilayer membrane is brought into contact with a protein or polypeptide according to step iii) in a solution of hydrochloric acid (HCl) at a pH between 2 and 4.

10. The method according to claim 8, wherein the multilayer membrane is brought into contact with a protein or polypeptide according to step iii) in a solution of sodium chloride (NaCl) at a pH between 5 and 7.

11. The method according to claim 8, further comprising an intermediate step consisting of equilibration of the multilayer membrane detached according to step ii) with a solution of hydrochloric acid (HCl) at a pH between 2 and 4 or of sodium chloride (NaCl) at a pH between 5 and 7 before step iii) is carried out.

12. The patch as claimed in claim 1, obtainable by a method as defined in claim 8.

13. The patch according to claim 1, wherein the protein is ovalbumin.

14. The patch according to claim 4, wherein the GAG or salt thereof is hyaluronic acid or sodium hyaluronidate.
